(19) United States
(12) Plant Patent Application Publication
(10) Pub. No.: US 2003/0115652 P1
(43) Pub. Date: Jun. 19, 2003

(54) Variety of geranium plant named 'Penjul'

(76) Inventor: Andrea Michalik, Dresden (DE)

Correspondence Address: Russell D. Orkin, 700 Koppers Building, 436 Seventh Avenue, Pittsburgh, PA, 15219-1818, US

(21) Appl. No.: 10/023,357

(22) Filed: Dec. 17, 2001

(51) Int. Cl. A01H 5/00

(52) U.S. Cl. PLT/330

(57) ABSTRACT

A new and distinct early flowering zonal geranium plant with red colored petals, dark foliage, compact and vigorous growth, and strong peduncles.

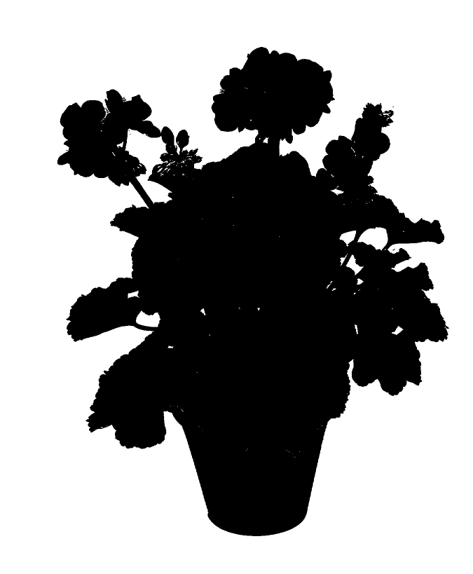

### BACKGROUND OF THE INVENTION

The present invention comprises a new and distinct cultivar of Pelargonium×hortorum known by the varietal name ‘Penjul’. The new variety was discovered as a seedling in a greenhouse in a selected breeding program in Dresden, Germany. The new variety is a selection from the cross of a set of fourteen proprietary unpatented breeding seedlings. The foliage color and habit of the new variety are similar to its parents but with different color intensity, flower shape, and cuttings yield. The new variety was first asexually reproduced in 1996 by cuttings in Dresden, Germany. The new variety has been trial and field tested in Dresden and has been found to retain its distinctive characteristics and remain true to type through successive propagations.

### DESCRIPTION OF THE DRAWING

The accompanying photographic drawing illustrates the new variety, with the color being as nearly true as is possible with color illustrations of this type.

### DESCRIPTION OF THE PLANT

The following detailed description sets forth the characteristics of the new cultivar. The data which defines these characteristics were collected by asexual reproductions carried out in Dresden, Germany of a potted plant grown in a warm greenhouse. The color readings were taken on a plant indoors under natural light. Color references are primarily to the R.H.S. Colour Chart of The Royal Horticultural Society of London.

### PLANT

Classification:

Botanical.—Pelargonium×hortorum.

Commercial.—Zonal.

Form: Upright.

Height: 25 cm from soil to top of foliage; 40 cm from soil to top of umbels.

Growth: Erect, well-branched.

Strength: Does not require artificial support.

Foliage: Zone present; arrangement is opposite.

Time to develop roots: 3.5 weeks.

Description of roots: Yellow Group 11D; adventive.

Leaves:

Size.—Length: 5 cm. Width: 6-7 cm.

Shape.—Overall: Kidney-shaped. Base: Open. Apex: Pointed.

Margin.—Bi-crenate.

Texture.—Hairy.

Color.—Upper surface: Yellow-Green Group 147A. Lower surface: Yellow-Green Group 147B.

Ribs and veins.—Venation: Net-like. Color: Green Group 138B.

Petioles:

Color.—Green Group 138B.

Length.—5 cm.

Stem:

Length.—20 cm.

Color.—Green Group 138B.

Internode length.—3-3.5 cm.

Lateral branch length.—19 cm.

### THE BUD

Shape when closed, just beginning to open:

Overall.—Umbel-shaped.

Individual bud.—Shell-shaped.

Size:

Size of individual bud.—Length: 1.5-1.8 cm. Width: 0.8-1.0 cm.

Size of cluster.—6-9 cm in diameter

Color: Red Group 40B.

### INFLORESCENCE

Blooming habit: Umbel-shaped.

Size:

Width.—10-12 cm.

Depth.—8 cm.

Borne: Flowers on umbel; umbel on pedicel; pedicel on peduncle.

Number of flowers per umbel: 25-30.

Flowers:

Form.—Zygomorphic.

Size.—Diameter: 3.5-4 cm. Depth: 1.0-1.3 cm.

Petals.—Color: Upper surface: Red Group 40A. Lower surface: Red Group 40B. Shape: Overall: Rounded to oval. Apex: Rounded. Base: Pointed. Size: Length: 2.4 cm. Width: 2.1 cm. Margin: Entire. Number per flower: 9-11. Texture: Smooth.

Tonality from a distance.—Shiny/scarlet.

Sepals:

Color.—Upper surface: Greyed-Orange Group 176A. Lower surface: Greyed-Orange Group 176A.

Shape.—Overall: Pointed; narrow. Apex: Pointed. Base: Wider and not pointed.

Size.—Length: 1.0-1.3 cm. Width: 0.3-0.4 cm.

Petaloids:

Quantity.—3-4.

Shape.—Irregular, smaller than petals.

Color.—Red Group 40A.

Pedicel:

Length.—3.5 cm.

Color.—Greyed-Orange Group 176A.

Peduncle:

Length.—13 cm.

Color.—Greyed-Orange Group 177A.

Disease/pest resistance: No unusual susceptibility to diseases or pests has been noted to date.

Flowering: Plant blooms until frost.

Natural flowering season: Summer in Germany.

### REPRODUCTIVE ORGANS

Stamens:

Anthers.—3 mm long.

Filaments.—Length: 0.4 cm. Color: Red Group 51A.

Pollen.—Orange Group 25A; abundant.

Pistils:

Number.—Five; divided.

Length.—0.3-0.4 cm.

Stigma color.—Red Group 47A.

Style.—Length: 0.6-0.7 cm. Color: Red-Purple Group 69C.

Ovaries.—Superior; hairy; 0.3 cm long; Green Group 140C.

Seeds: Rarely present; if present, 5 per flower maximum, 0.3-0.4 cm, Greyed-Orange Group 165A.

### GENERAL CHARACTERISTICS

‘Penjul’ has the following general characteristics.

1. Compact growth.

2. Vigorous growth habit.

3. Intense and stable flower color.

4. Stable and upright peduncles.

5. Fast rooting.

6. Suitable for bedding.

7. Stable outside.

8. Dark foliage.

9. Long lasting.

10. Suitable for transport.

I claim:

1. A new and distinct variety of geranium plant substantially as shown and described.
